(19) United States
(12) Patent Application Publication
(10) Pub. No.: US 2005/0288581 A1
(43) Pub. Date: Dec. 29, 2005

(54) Acoustic coupling gel for combined mammography and ultrasound image acquisition and methods thereof

(76) Inventors: Ajay Kapur, Clifton Park, NY (US); Kai Erik Thomenius, Clifton Park, NY (US)

Correspondence Address: Patrick S. Yoder;FLETCHER YODER, P.O. Box 692289, Houston, TX, 77269-2289, US

(21) Appl. No.: 10/879,458

(22) Filed: Jun. 29, 2004

(51) Int. Cl. A61B 8/00; A61B 6/04

(52) U.S. Cl. 600/437; 128/915; 378/37

(57) ABSTRACT

In accordance with embodiments of the present technique, a combined mammography and ultrasound imaging system is provided. The system includes an ultrasound probe, which transmits ultrasound signals to a breast of a patient and receives reflected ultrasound signals from the breast. The system further includes a first acoustic coupling sheath. A first side of the first acoustic coupling sheath is coupled to a face of the ultrasound probe. The system also includes a mammography compression plate for compressing the breast of the patient. A second acoustic coupling sheath coupled to a side of the mammography compression plate contacts the breast of the patient.

This invention was made with Government support under contract number RO1 CA91713 01A1 awarded by the United States National Institutes of Health. The Government has certain rights in the invention.

### BACKGROUND

The present invention relates generally to an image acquisition technique and specifically to acoustic coupling gels and devices, such as for co-registration of mammography and ultrasound images.

In modern healthcare facilities, medical diagnostic and imaging systems are used for identifying, diagnosing, and treating diseases. Diagnostic imaging refers to any visual display of structural or functional patterns of organs or tissues for a diagnostic evaluation. Currently, a number of modalities exist for medical diagnostic and imaging systems. These include, for example, ultrasound systems, X-ray imaging systems (including mammography system), molecular imaging systems, computed tomography (CT) systems, positron emission tomography (PET) systems and magnetic resonance imaging (MRI) systems.

One known imaging technique is mammography, by which a breast of a patient may be non-invasively examined or screened to detect abnormalities, such as lumps, fibroids, lesions, calcifications, and so forth. Typically mammography employs specialized radiographic techniques to generate images representative of a breast tissue. A mammography imaging system typically comprises an X-ray imaging system, which uses a source of radiation, such as an X-ray source, a breast-positioning sub-system, an X-ray detector for imaging, data acquisition computers, control software and display monitors. X-ray imaging is generally very effective for detailed characterization of benign and cancerous structures such as calcifications and masses embedded in the breast tissue.

Another known imaging technique is ultrasound. An ultrasound imaging system uses an ultrasound probe for transmitting ultrasound signals into an object, such as the breast of the patient being imaged, and for receiving reflected ultrasound signals there from. The reflected ultrasound signals received by the ultrasound probe are processed to reconstruct an image of the object. Ultrasound imaging is very effective at other types of diagnosis, such as for differentiating benign cysts and masses.

Co-registered mammography and ultrasound image acquisition is a technique wherein dual modality images are acquired with the patient in virtually the same position within a single examination so that the duel modality images (image sets) are intrinsically registered to one another. When the breast of the patient is in a compressed state, image data is generated by the ultrasound imaging system and is combined with data from the X-ray imaging system to leverage strengths of both techniques.

In an ultrasound imaging system, an ultrasonic coupling gel or paste is typically used to ensure proper contact between the ultrasound probe of the ultrasound system and skin of the patient being imaged. Ultrasound gels generally ensure good transmission of acoustic energy, but have certain drawbacks, particularly in applications such as multi-modality mammography. For example, they can be messy to apply, and generally require subsequent clean up of both the patient and imaging equipment (e.g. the ultrasound probe) that may come into contact with the gel during an imaging session. Further a non-uniform application of the coupling gel could easily lead to a sub-optimal image quality.

Thus, there exists a need for a new technique for providing acoustic coupling between the ultrasound probe and an object to be imaged. There is a particular need for a technique that is compatible for mammography and ultrasound imaging techniques for co-registered mammography and ultrasound image data acquisition.

### BRIEF DESCRIPTION

Briefly, in accordance with one aspect of present invention, a combined mammography and ultrasound imaging system is provided. The system includes an ultrasound probe, which transmits ultrasound signals to a breast of a patient and receives reflected ultrasound signals from the breast. The system further includes a first acoustic coupling sheath. A first side of the first acoustic coupling sheath is coupled to a face of the ultrasound probe. The system also includes a mammography compression plate for compressing the breast of the patient. A second acoustic coupling sheath coupled to a side of the mammography compression plate contacts the breast of the patient.

A method of conducting a mammography examination is also provided. The method generally includes coupling an acoustic coupling sheath to a lower surface of a mammography compression plate. An object, like a breast of a patient, is then compressed between an X-ray detector and the acoustic coupling sheath via the mammography compression plate. Image data is acquired for reconstruction of mammography image.

In a variation of the invention, the method includes coupling a first side of a first acoustic coupling sheath to a face of an ultrasound probe. The method also includes coupling a second acoustic coupling sheath to a lower surface of a mammography compression plate. Then an object, like a breast of a patient, is compressed between an X-ray detector and the second acoustic sheath and via the mammography compression plate. The method further includes acquiring ultrasound image data by transmitting acoustic energy through the first acoustic coupling sheath, the mammography compression plate and the second acoustic coupling sheath to the compressed breast.

A method of acquiring a co-registered mammography and ultrasound image is also provided. The method includes coupling a first side of a first acoustic coupling sheath to a face of an ultrasound probe. The method also includes coupling a second acoustic coupling sheath to a lower surface of a mammography compression plate. An object, like a breast of a patient to be imaged is compressed between the second acoustic coupling sheath and an X-ray detector via the mammography compression plate. Mammography image data is acquired for reconstruction into a mammography image and for further analysis. Then ultrasound image data is acquired by transmitting acoustic energy through the first acoustic coupling sheath, the second acoustic coupling sheath and the mammography compression plate in conjunction with the analysis based on the mammography image.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatic representation of one embodiment of a mammography imaging system in accordance with aspects of present technique;

FIG. 2 is a diagrammatic representation of one embodiment of an ultrasound imaging system in accordance with aspects of present technique;

FIG. 3 is a partial side elevation of an exemplary arrangement for an ultrasound examination of a breast of a patient, showing acoustic coupling sheaths and a mammography compression plate in accordance with aspects of present technique;

FIG. 4 is a side view of an exemplary embodiment of an acoustic coupling sheath for an ultrasound probe in accordance with aspects of present technique;

FIG. 5 is a side view of another exemplary embodiment of an acoustic coupling sheath for an ultrasound probe in accordance with aspects of present technique;

FIG. 6 is a plan view of another exemplary embodiment of an acoustic coupling sheath for an ultrasound probe in accordance with aspects of present technique;

FIG. 7 is a side view of an exemplary embodiment of an acoustic coupling sheath for a mammography compression plate in accordance with aspects of present technique;

FIG. 8 is a side view of another exemplary embodiment of an acoustic coupling sheath for a mammography compression plate in accordance with aspects of present technique;

FIG. 9 is a top view of another exemplary embodiment of an acoustic coupling sheath for a mammography compression plate in accordance with aspects of present technique;

FIG. 10 is a flowchart illustrating exemplary process steps for acquiring mammography images in accordance with aspects of present technique;

FIG. 11 is a flowchart illustrating exemplary process steps for acquiring ultrasound images in accordance with aspects of present technique; and

FIG. 12 is a flowchart illustrating exemplary process steps for acquiring co-registered mammography and ultrasound images in accordance with aspects of present technique.

### DETAILED DESCRIPTION

The present technique is directed towards co-registration of mammography and ultrasound images and specifically to acoustic coupling gels for such co-registration. In general, the co-registration technique employs a mammography imaging system and an ultrasound imaging system. As will be appreciated by those of ordinary skill in the art, the present techniques may also be applied in other medical and non-medical contexts.

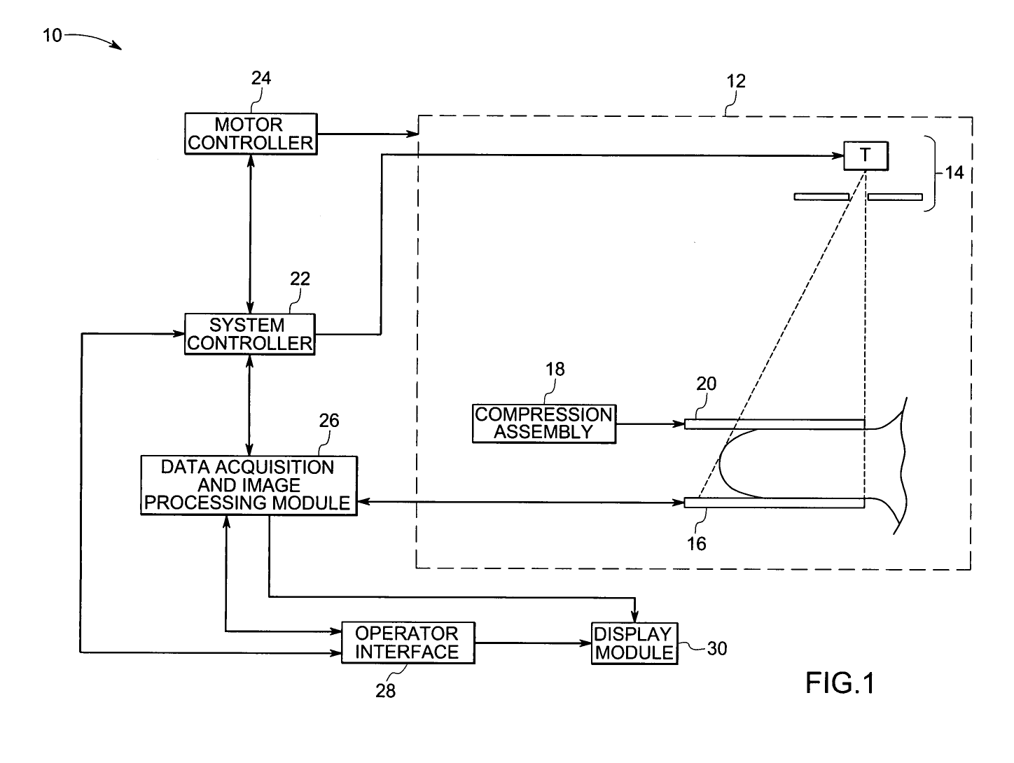

Turning now to the drawings, and referring first to FIG. 1, an exemplary mammography imaging system 10 is illustrated diagrammatically in conjunction with the present technique. As depicted, the mammography imaging system 10 includes an image data acquisition system 12. The image data acquisition system 12 includes an X-ray source 14, an X-ray detector 16 and a compression assembly 18. The mammography imaging system 10 further includes a system controller 22, a motor controller 24, data acquisition and image-processing module 26, an operator interface 28 and a display module 30.

The X-ray source 14 further comprises an X-ray tube and a collimator configured to generate a beam of X-rays when activated. The X-ray tube is one example of the X-ray source 14. Other types of the X-ray sources 14 may include some or all of emitters of a solid state X-ray source. The X-ray source 14 may be movable in one, two or three dimensions, either by manual or by automated means. The image data acquisition system 12 may move the X-ray source 14 via tracks, ball-screws, gears, belts, and so forth. For example, the X-ray source 14 may be located at an end of a mechanical support, such as a rotating arm or otherwise adjustable support, which may be moved by the image data acquisition system 12.

The X-ray detector 16 may be stationary, or may be configured to move either independently or in synchrony with the X-ray source 14. In a present embodiment, the X-ray detector is a digital flat panel detector. The image data acquisition system 12 may move the X-ray detector 16 via tracks, ball-screws, gears, belts, and so forth. The X-ray detector also provides support an object, such as a breast of a patient to be imaged in the mammography application described below.

The compression assembly 18 is configured to compress the patient breast to near uniform thickness against the X-ray detector 16 for performing mammography imaging. The compression assembly 18 comprises a mammography compression plate 20, which may be a flat, inflexible plate, or alternative compression device. The mammography compression plate 20 is configured to be radiolucent to transmit X-rays and is further configured to be sonolucent to transmit ultrasound signals. The compression assembly 18 may be used to acquire co-registered mammography, ultrasound images, tomosynthesis X-ray images and Doppler images.

The system controller 22 controls operation of the image data acquisition system 12 that provides for a physical motion required by the X-ray source 14 and/or the X-ray detector 16. Movement is, in turn controlled through the motor controller 24 in accordance with an imaging trajectory for use in mammography. Therefore, by means of the image data acquisition system 12, the system controller 22 may facilitate acquisition of radiographic projections at various angles through a patient. The system controller 22 further controls an activation and operation of other components of the system, including collimation of the X-ray source 14. Moreover, the system controller 22 may be configured to provide power and timing signals to the X-ray source 14. The system controller 22 may also execute various signal processing and filtration functions. In general, the system controller 22 commands operation of the mammography imaging system 10 to execute examination protocols and to acquire resulting data.

The system controller 22 controls the data acquisition and image-processing module 26. The data acquisition and image-processing module 26 communicates with the X-ray detector 16 and typically receives data from the X-ray detector 16, such as a plurality of sampled analog signals or digitized signals resulting from exposure of the X-ray detector to X-rays. The data acquisition and image-processing module 26 may convert the data to digital signals suitable for processing.

The operator interface 28 may include a keyboard, a mouse, and other user interaction devices. The operator interface 28 can be used to customize settings for the mammography imaging and for affecting system level configuration changes. The operator interface 28 is connected to the data acquisition and image-processing module 26, the system controller 22 and the display module 30. The display module 30 presents a reconstructed image of an object within a region of interest based on data from the data acquisition and image-processing module 26. As will be appreciated by those skilled in the art, digitized data representative of individual picture elements or pixels is processed by the data acquisition and image-processing module to reconstruct the desired image. The image data, in either raw or processed forms, may be stored in the system or remotely for later reference and image reconstruction.

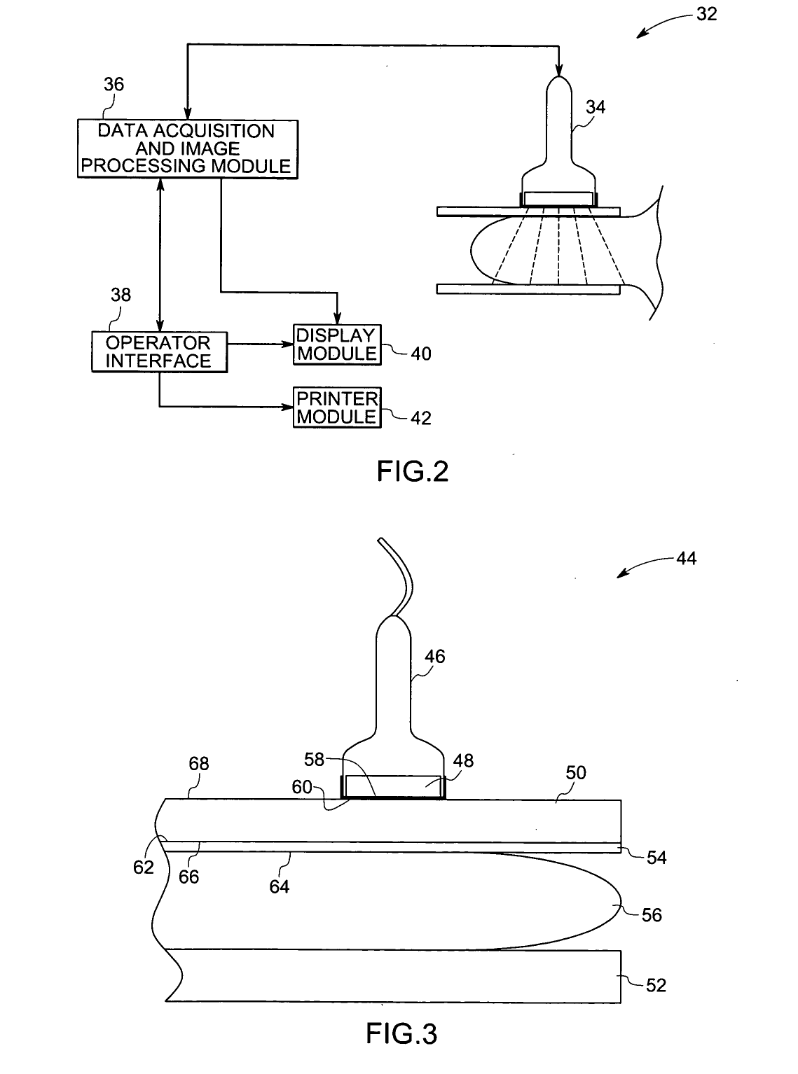

FIG. 2 illustrates an exemplary ultrasound imaging system 32 for use in conjunction with the present technique. As depicted, the ultrasound imaging system 32 includes an ultrasound probe 34, a data acquisition and image-processing module 36, which includes beam-formers, an operator interface 38, a display module 40 and a printer module 42. In a hybrid imaging system based upon both X-ray and ultrasound techniques, certain of these components or modules may be partially or fully integrated to perform image acquisition and processing from both systems.

The ultrasound imaging system 32 uses the ultrasound probe 34 for transmitting a plurality of ultrasound signals into an object, such as a breast of a patient being imaged, and for receiving a plurality of reflected ultrasound signals there-from. The ultrasound probe 34, according to aspects of present technique, includes at least one of an ultrasound transducer, a piezoelectric crystal, an opto-acoustic transducer and a micro electromechanical system device. As will be appreciated by those of ordinary skill in the art, the plurality of reflected ultrasound signals from the object carry information about thickness, size, and location of various tissues, organs, tumors, and anatomical structures in relation to transmitted ultrasound signals. The plurality of reflected ultrasound signals received by the ultrasound probe 34 are processed for constructing an image of the object. In certain embodiments, the ultrasound probe 34 can be hand-held or mechanically positioned using a robotic assembly.

The data acquisition and image-processing module 36 sends signals to and receives information from the ultrasound probe 34. Thus, the data acquisition and image-processing module 36 controls strength, width, duration, and a frequency of the plurality of ultrasound signals transmitted by the ultrasound probe 34, and decodes the information contained in the plurality of reflected ultrasound signals from the object to a plurality of discernable electrical and electronic signals. Once the information is obtained, an ultrasound image of the object located within a region of interest of the ultrasound probe 34 is reconstructed in accordance with generally known reconstruction techniques.

The operator interface 38 may include a keyboard, a mouse, and other user interaction devices. The operator interface 38 can be used to customize a plurality of settings for an ultrasound examination, and for effecting system level configuration changes. The operator interface 38 is connected to the data acquisition and image-processing module 36, the display module 40 and to the printer module 42. The display module 40 receives information from the data acquisition and image-processing module 36 and presents the image of the object within the region of interest of the ultrasound probe 34. The printer module 42 is used to produce a hard copy of the ultrasound image in either gray-scale or color. As noted above, some or all of these system components may be integrated with those of the X-ray system described above.

FIG. 3 illustrates an exemplary arrangement 44 for an ultrasound examination as part of acquiring co-registered mammography and ultrasound images in accordance with aspects of present technique. The exemplary arrangement comprises an ultrasound probe 46, a first acoustic coupling sheath 48, a mammography compression plate 50, an X-ray detector 52 and a second acoustic coupling sheath 54. Each of these components is described in further detail below.

The ultrasound probe 46 is configured to transmit and receive ultrasound signals. The first acoustic coupling sheath 48 comprises a layer of adhesive on a first side 58. The first acoustic sheath is coupled to a face 60 of the ultrasound probe 46. The first acoustic coupling sheath 48 is further configured to act as an ultrasonic acoustic coupling to ensure proper contact for transmission of the ultrasound signals. The mammography compression plate 50 is sonolucent and radiolucent and is capable of transmitting both ultrasound signals and X-rays. The mammography compression plate 50 is primarily used for compressing the breast of the patient in combination with the X-ray detector 52 to a near uniform thickness for acquiring mammography images.

The second acoustic coupling sheath 54 is also sonolucent and radiolucent and is capable of transmitting both ultrasound signals and X-rays. The second acoustic coupling sheath 54 comprises layers of adhesive on a first side 62 and on a second side 64. The second acoustic coupling sheath may be of any suitable size to cover the entire breast, or only a portion thereof, as desired for the anatomical structures to be imaged. The second acoustic coupling sheath 54 is coupled to the mammography compression plate 50. The first side 62 of the second acoustic coupling sheath 54 abuts a lower surface 66 of the mammography compression plate 50, while the second side 64 is configured to contact a breast of a patient 56 to ensure proper contact for transmission of the ultrasound signals. The ultrasound probe 46, along with the first acoustic coupling sheath 48 moves over a top surface 68 of the mammography compression plate 50 either manually or mechanically to transmit ultrasound signals. As appreciated by those skilled in the art, a moistening agent or a wetting agent such as water or glycol may be applied over the top surface 68, i.e., between the ultrasound probe and the mammography compression plate, to reduce friction and improve wetting characteristics. During the ultrasound portion of a breast examination, then, the ultrasound signals are transmitted through the first acoustic coupling sheath 48, the mammography compression plate 50, the second acoustic coupling sheath 54 to the breast 56 and to receive reflected ultrasound signals there from to acquire images of the breast 56. It can be noted that the mammography compression plate enables exerting uniform and sufficient pressure on the breast for medical examinations.

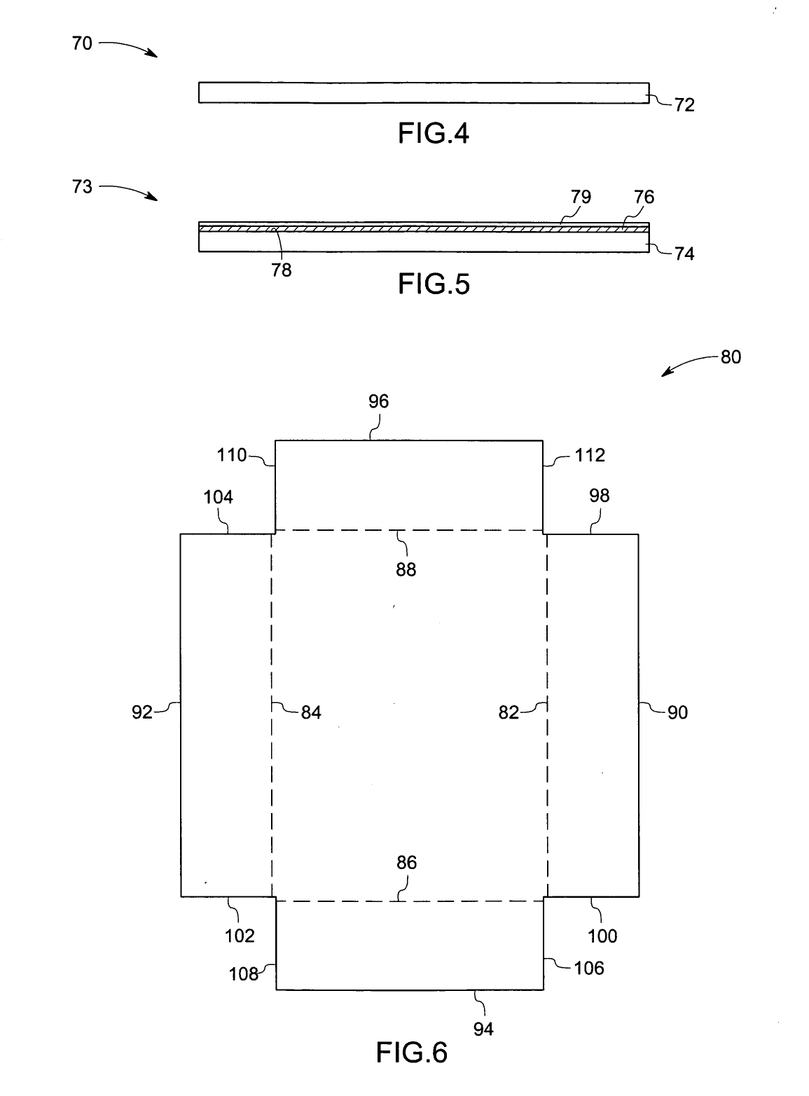

FIG. 4 illustrates an exemplary embodiment of an acoustic coupling sheath 70 configured to couple with a face of an ultrasound probe to ensure proper contact for transmission of ultrasound signals. The acoustic coupling sheath 70 may comprise a body 72 made of a sonolucent material with a thickness of about 0.1 cm.

FIG. 5 illustrates another embodiment of an acoustic coupling sheath 73 configured to couple with a face of an ultrasound probe to ensure proper contact for transmission of ultrasound signals. The acoustic coupling sheath 73 may also comprise a body 74 with a thickness of about 0.1 cm made of a sonolucent material and a layer of adhesive 76 on a first side 78 to ensure a uniform coupling with the face of the ultrasound probe. Reference numeral 79 represents a peal-off cover to protect the layer of adhesive 76. The peal-off cover 79 is removed just before fixing the acoustic coupling sheath 73 to the ultrasound probe. In general, the body 74 may include a base material or carrier impregnated with an acoustic gel. The carrier and acoustic gel facilitate the transmission of ultrasound energy in a manner generally similar to conventional acoustic gels used in ultrasound applications, while facilitating setup and cleaning of the ultrasound probe, as described below.

FIG. 6 illustrates another exemplary embodiment of an acoustic coupling sheath 80. The acoustic coupling sheath 80 comprises folding lines 82, 84, 86 and 88. The acoustic coupling sheath 80 further comprises edges 90 and 92 with a length of around 4.5 cm, edges 94 and 96 with a length of around 1.0 cm, edges 98, 100, 102 and 104 with a length of around 1.5 cm, edges 106, 108, 110 and 112 with a length of around 1.3 cm. A distance between folding line 82 and edge 90 may be around 1.0 cm. Similarly a distance between folding line 84 and edge 92 may be around 1.0 cm. A distance between folding line 86 and edge 94 may be around 1.0 cm. Similarly a distance between folding line 88 and edge 96 may be around 1.0 cm. An open box-like arrangement may thus be formed of the acoustic coupling sheath 80 using the folding lines and the edges as explained above to facilitate easy coupling with a face of an ultrasound probe. The edges 90, 92, 94 and 96 may adhere to sides of ultrasound probe as depicted in FIG. 3. The various dimensions of the acoustic coupling sheath mentioned above are exemplary only. It may be noted that these dimensions of the acoustic coupling sheath may vary for use with different ultrasound probes of different sizes, shapes, configurations and so forth.

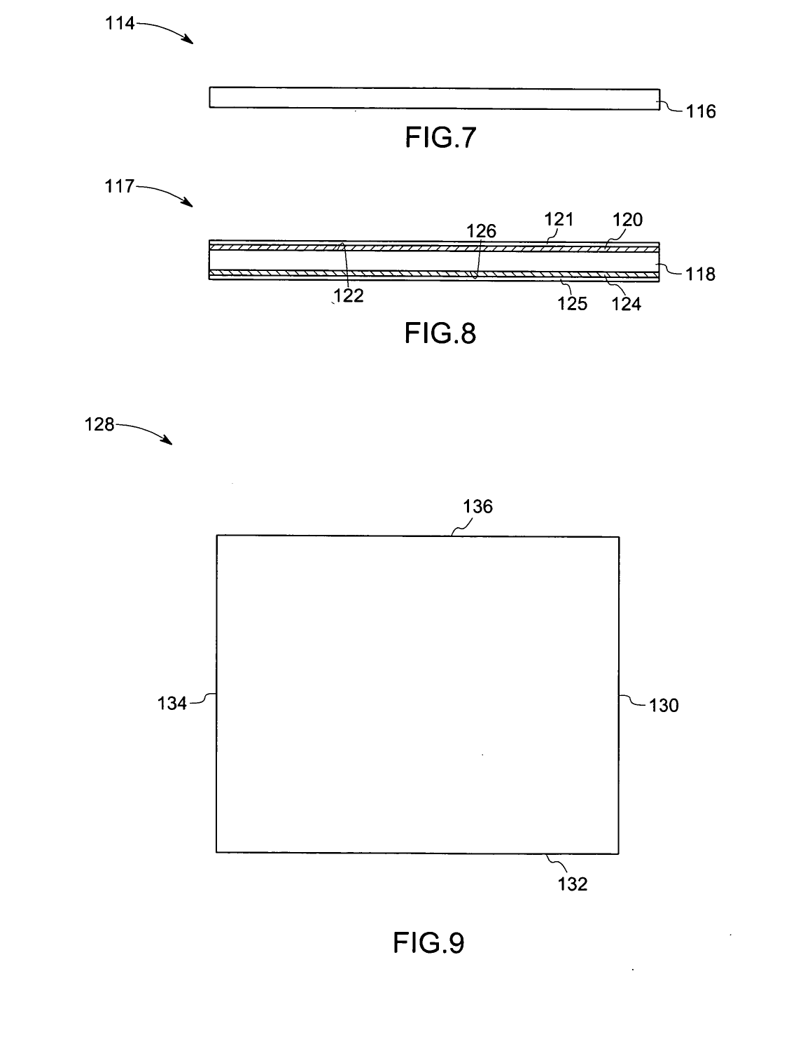

FIG. 7 illustrates an exemplary embodiment of an acoustic coupling sheath 114 configured to transmit both ultrasound signals and X-rays. The acoustic coupling sheath 114 is configured to couple with a lower surface of a mammography compression plate and to contact a breast of a patient. The acoustic coupling sheath 114 may comprise a body 116 made of a sonolucent and radiolucent material with a thickness of about 0.1 cm.

FIG. 8 illustrates another exemplary embodiment of an acoustic coupling sheath 117 configured to transmit both ultrasound signals and X-rays. The acoustic coupling sheath 117 may also comprise a body 118 made of a sonolucent and radiolucent material with a thickness of about 0.1 cm and a layers of adhesive 120 on a first side 122 and a layer of adhesive 124 on a second side 126 to ensure a uniform coupling with the lower face of the mammography compression plate and to ensure a uniform contact with the breast. As explained above, reference numerals 121 and 125 represent peal-off covers to protect the layers of adhesive 120 and 124 respectively. The peal-off covers 121 and 125 will be removed just before using the acoustic coupling sheath 117 for medical procedures. As in the case of probe sheath, sheath 117 may include a body or carrier that is impregnated with an acoustic gel to facilitate transmission of acoustic energy.

FIG. 9 illustrates another exemplary embodiment of an acoustic coupling sheath 128 configured to transmit both ultrasound signals and X-rays with a thickness of about 0.1 cm comprises edges 130, 132, 134 and 136. As can be noted that the acoustic coupling sheath may be selected with any suitable size to cover the entire breast or a portion thereof.

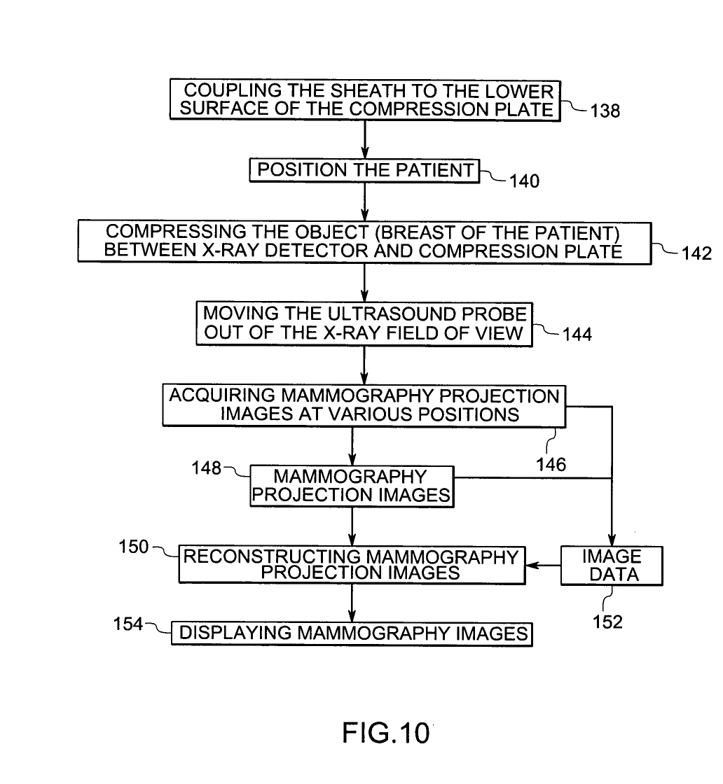

FIG. 10 illustrates exemplary process steps for acquiring mammography images using a combined mammography and ultrasound imaging system in accordance with aspects of the present technique. The process generally begins with coupling a radiolucent and sonolucent sheath to a lower surface of a mammography compression plate of mammography imaging system as in step 138. Then a patient may be positioned for examination as in step 140. A breast of the patient, thus positioned beneath the plate is compressed for imaging between the mammography compression plate and an X-ray detector as in step 142. An ultrasound probe is moved out of the X-ray field of view to facilitate X-ray imaging as in step 144. In a presently contemplated embodiment, the physical components of the ultrasound system may be pivoted or swung from the path of X-rays during this portion of the examination. Mammography projection images are then acquired at plurality of positions of an X-ray source as in step 146. The mammography projection images 148 thus acquired are used for reconstructing mammography images as in step 150 based on image data 152. The image data 152 comprises digitized pixel data that can be interpreted for reconstruction of useful images. The reconstructed mammography images will then be displayed as in step 154 for analysis.

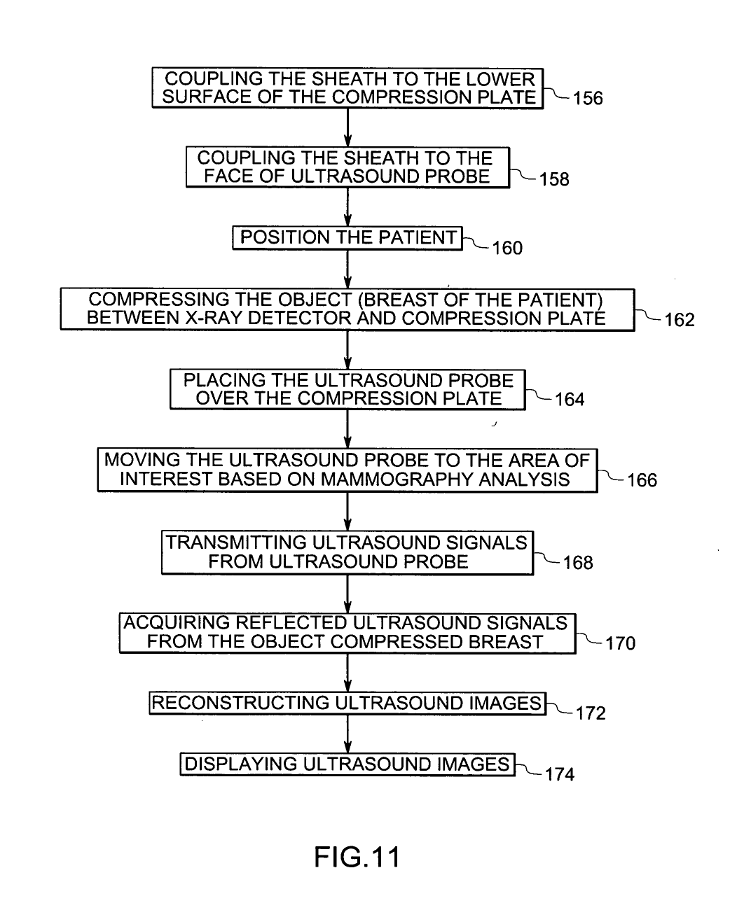

FIG. 11 illustrates an exemplary method for acquiring ultrasound images using a combined mammography and ultrasound imaging system in accordance with aspects of the present technique. In the method of FIG. 11, a radiolucent and sonolucent sheath is coupled to a lower surface of a mammography compression plate of mammography imaging system as in step 156. A sonolucent sheath is coupled to a face of an ultrasound probe to ensure proper transmission of ultrasound signals as in step 158. As described above, the patient is positioned for examination as in step 160. A breast of the patient to be imaged is compressed between the mammography compression plate and an X-ray detector as in step 162. The ultrasound probe along with the sonolucent sheath is placed over an upper surface of the mammography compression plate as in step 164. Then the ultrasound probe is moved close to an area of interest of the breast as in step 166. The ultrasound probe transmits ultrasound signals to the breast as in step 168. Ultrasound signals reflected from the breast are acquired as in step 170. The ultrasound signals thus acquired are used to reconstruct ultrasound images as in step 172. The reconstructed ultrasound images may then be displayed, as indicated at step 174.

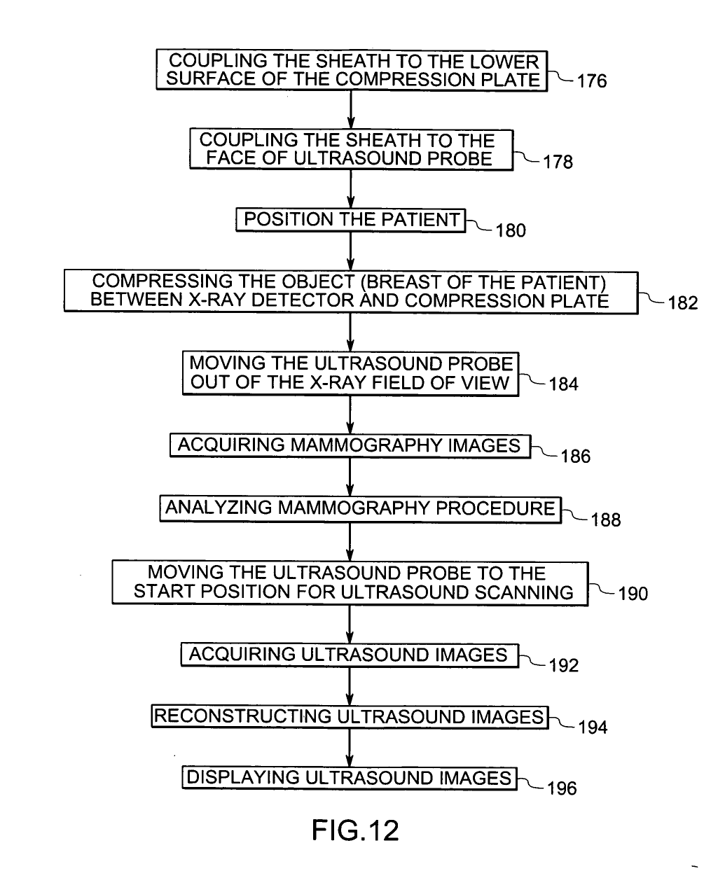

FIG. 12 illustrates an exemplary method for acquiring co-registered mammography and ultrasound images using a combined mammography and ultrasound imaging system in accordance with aspects of the present technique. In the exemplary method represented, a radiolucent and sonolucent sheath is coupled to a lower surface of a mammography compression plate of mammography imaging system as in step 176. A sonolucent sheath is coupled to a face of the ultrasound probe as in step 178. A patient is positioned for examination as in step 180. A breast of the patient is compressed between the mammography compression plate and an X-ray detector as in step 182. An ultrasound probe is moved out of the X-ray field of view as in step 184. Mammography images using an X-ray source are acquired as in step 186. The mammography images thus acquired are analyzed as in step 188. Then the ultrasound probe is moved to a start position for ultrasound scanning as in step 190. Ultrasound image data is acquired in accordance with the analysis based on the mammography images as in step 192. The ultrasound image data is reconstructed to form ultrasound image as in step 194. The reconstructed ultrasound image is then displayed as in step 196.

Based upon the X-ray and ultrasound image data acquired, combined image may be produced. That is, while separate images may be used for diagnostic and other purposes, the image processing circuitry may be configured to register X-ray images with ultrasound images for enhanced diagnostic purposes. In other situations, based upon features visible in either X-ray or ultrasound images, or both, additional images may be acquired during an examination via either the mammography imaging system or the ultrasound imaging system, or both.

It should be noted that, while use of the sheaths described above is particularly useful for the mammography application, such use is not limited to breast imaging. The probe sheath, in particular, may be used in many other applications. In general, both sheaths are designed to be disposable for single use. Application of the sheaths is facilitated by adhesive provided on one or both of their faces. Such adhesive may be of any suitable type, such as adhesives commonly used in medical applications for transmission of acoustic energy. It should also be noted that size and configuration of the sheaths, and particularly of the probe sheath, may be adapted for specific equipment. A number of different sheaths may thus be designed for probes made by various manufactures, as well as for specific probe models. Similarly, sheaths designed to interface a mammography compression plate with the patient tissue may be made in various sizes and configurations, allowing for selection by clinicians based upon the size of the tissue to be imaged.

It should also be noted that the sheaths may be made of homogeneous material. These sheaths impregnated with acoustic gel may include, for example, sheaths commercially available from Sonotech Inc of Bellingham, Wash., USA. As appreciated by those skilled in the art these sheaths provide low and uniform attenuation throughout mammography and ultrasound fields of view during medical examinations as opposed to conventional gels, the thickness and distribution of which can vary substantially.

Furthermore, some medical examinations require imaging the breast at different orientations. For example, during cranio-caudal (CC) imaging, the ultrasound imaging is carried out with the ultrasound probe being above the breast. During medial lateral oblique (MLO) imaging, the imaging system components (e.g. mammography compression plate and X-ray detector) may be oriented to 30 degrees, 45 degrees and up to 90 degrees. In such situations where the imaging system components may be positioned at various angles such as those for oblique views of the breast, the acoustic-coupling sheaths enhance ease of use over standard gels, which may be less effective due to insufficient viscosity of the gel. (i.e. causing running of the gel).

While only certain features of the invention have been illustrated and described herein, many modifications and changes will occur to those skilled in the art. It is, therefore, to be understood that the appended claims are intended to cover all such modifications and changes as fall within the true spirit of the invention.

1. A combined mammography and ultrasound imaging system comprising:
an ultrasound probe, wherein the ultrasound probe transmits ultrasound signals to a breast of a patient and receives reflected ultrasound signals there-from;
a first acoustic coupling sheath, wherein a first side of the first acoustic coupling sheath is coupled to a face of the ultrasound probe;
a mammography compression plate for compressing the breast of the patient; and
a second acoustic coupling sheath coupled to a side of the mammography compression plate and configured to contact the breast of the patient.

2. The system of claim 1, further comprising a wetting agent disposed intermediate the first acoustic sheath and the mammography compression plate.

3. The system of claim 1, wherein the first acoustic coupling sheath comprises a layer of adhesive on the first side.

4. The system of claim 1, further comprising
an X-ray source configured to move along an imaging trajectory; and
an X-ray detector, wherein the X-ray detector is configured to detect X-rays emitted by the X-ray source and to generate signals in response to the detected X-rays.

5. The system of claim 1, wherein the second acoustic coupling sheath is configured to substantially cover the compressed breast of the patient.

6. The system of claim 1, wherein the second acoustic coupling sheath is configured to be sonolucent and radiolucent.

7. The system of claim 1, wherein the second acoustic coupling sheath comprises of a layer of adhesive on a first side thereof configured to contact the mammography compression plate.

8. The system of claim 1, wherein the second acoustic coupling sheath comprises of a layer of adhesive on first and second sides thereof.

9. The system of claim 1, wherein the first acoustic coupling sheath and the second acoustic coupling sheath are made of substantially identical materials.

10. The system of claim 1, further comprising an ultrasound image processing system, wherein the ultrasound image processing system is configured to acquire the reflected ultrasound signals from the ultrasound probe and to reconstruct an ultrasound image based thereon.

11. The system of claim 1, further comprising an X-ray system controller, wherein the X-ray system controller is configured to operate an X-ray source.

12. The system of claim 1, further comprising a mammography image processing system, wherein the mammography image processing system is configured to acquire the signals from an X-ray detector and to reconstruct an image based thereon.

13. An acoustic coupling sheath for facilitating ultrasound examinations comprising a generally sheet-like body made of an acoustically conductive material, the body having a first side, dimensionally configured to be coupled to a face of an ultrasound probe, and a second side opposite to the first side and configured to contact a surface through which ultrasound energy is to be transmitted.

14. The acoustic coupling sheath of claim 13, wherein the first side comprises a layer of adhesive.

15. The acoustic coupling sheath of claim 13, wherein the second side comprises a layer of adhesive.

16. The acoustic coupling sheath of claim 13, is dimensionally configured to wrap at least partially around the face of the ultrasound probe.

17. The second acoustic coupling sheath of claim 13, is configured to be sonolucent and radiolucent.

18. An acoustic coupling sheath for performing ultrasound examination comprising a generally sheet-like body made of an acoustically conductive material, the body having a first side configured to contact a side of a mammography compression plate, and a second side configured to contact a breast of a patient.

19. The acoustic coupling sheath of claim 18, wherein the first side comprises of a layer of adhesive.

20. The acoustic coupling sheath of claim 18, wherein the first and the second sides each comprise of a layer of adhesive.

21. The acoustic coupling sheath of claim 18, is configured to be sonolucent and radiolucent.

22. A method for conducting a mammography examination, the method comprising:
coupling an acoustic coupling sheath to a lower surface of a mammography compression plate;
compressing a breast of a patient to be imaged between the acoustic coupling sheath and an X-ray detector via the mammography compression plate; and
acquiring image data for reconstruction into a mammography image.

23. The method of claim 22, wherein acquiring image data comprises:
moving an X-ray source along an imaging trajectory;
emitting X-rays from the X-ray source at a plurality of locations on the imaging trajectory;
generating signals from an X-ray detector by detecting X-rays emitted by the X-ray source; and
acquiring signals from the X-ray detector for reconstruction of the mammography image.

24. A method for conducting an ultrasound examination, the method comprising:
coupling a first side of a first acoustic coupling sheath to a face of an ultrasound probe;
coupling a second acoustic coupling sheath to a lower surface of a mammography compression plate;
compressing a breast of a patient to be imaged between the second acoustic coupling sheath and an X-ray detector via the mammography compression plate; and
acquiring ultrasound image data by transmitting acoustic energy through the first acoustic coupling sheath, the mammography compression plate and the second acoustic coupling sheath.

25. The method of claim 24, further comprising constructing ultrasound images comprises:
moving the ultrasound probe on an upper surface of the mammography compression plate;
transmitting ultrasound signals through the first acoustic coupling sheath, the mammography compression plate and the second acoustic coupling sheath to the breast of the patient; and
acquiring reflected ultrasound signals from the breast and constructing the ultrasound images.

26. A method for acquiring co-registered mammography and ultrasound images, the method comprising:
coupling a first side of a first acoustic coupling sheath to a face of an ultrasound probe;
coupling a second acoustic coupling sheath to a lower surface of a mammography compression plate;
compressing a breast of a patient to be imaged between the second acoustic coupling sheath and an X-ray detector via the mammography compression plate;
acquiring image data for reconstruction into a mammography image;
analyzing the mammography image to generate diagnostic data; and
acquiring ultrasound image data by transmitting acoustic energy through the first acoustic coupling sheath, the mammography compression plate and the second acoustic coupling sheath in conjunction with diagnostic data based on the mammography image.

27. The method of claim 26, wherein acquiring ultrasound images comprises:
transmitting ultrasound signals through the first acoustic coupling sheath, the mammography compression plate and the second acoustic coupling sheath to the breast of the patient; and
acquiring reflected ultrasound signals from the breast and constructing the ultrasound images.
